(19) United States
(12) Patent Application Publication
(10) Pub. No.: US 2024/0293622 A1
(43) Pub. Date: Sep. 5, 2024

(54) Autoinjector

(71) Applicant: Becton Dickinson France, Le Pont-de-Claix (FR)

(72) Inventors: Michael Fiard, Eybens (FR); Rémi Jamon, Vaulnaveys le Haut (FR); Louis Petit, Venon (FR); Pierre Ducarouge, Grenoble (FR); Arnaud Murier, Grenoble (FR)

(21) Appl. No.: 18/572,984

(22) PCT Filed: Jun. 21, 2022
(86) PCT No.: PCT/EP2022/066912

§ 371 Date: Dec. 21, 2023

(30) Foreign Application Priority Data
Jun. 25, 2021 (EP) 21305881.1

(51) Int. Cl. A61M 5/315; A61M 5/32

(52) U.S. Cl. CPC A61M 5/31501; A61M 5/3243; A61M 2005/3151

(57) ABSTRACT

The autoinjector has a proximal end and, a longitudinal axis A, and a housing configured to receive a medical container having a barrel defining a reservoir for containing a medical product, and said barrel having a distal end provided with a needle and an opened proximal end configured to receive a plunger rod for pushing a stopper arranged inside the barrel. A needle cover is coupled to and movable with respect to said housing between a first extended position, a retracted position, and a second extended position wherein the needle cover moves back in the distal direction to shield the needle. An injection mechanism is configured to move the plunger rod distally inside the barrel in order to expel the medical product contained inside the barrel. A holder is movable with respect to the housing between a passive position wherein the holder does not trigger the injection mechanism and an active position wherein the holder triggers the injection mechanism. The autoinjector includes blocking means configured to prevent the medical container from moving proximally inside the housing, such that the medical container cannot displace the holder towards the active position if the autoinjector falls down onto a floor, proximal end first.

### CROSS-REFERENCE TO RELATED APPLICATIONS

This application is the United States national phase of International Application No. PCT/EP2022/066912 filed Jun. 21, 2022 and claims priority to European Patent Application No. 21305881.1 filed Jun. 25, 2021, the disclosures of which are hereby incorporated by reference in their entireties.

### BACKGROUND OF THE INVENTION

### Field of the Invention

The present invention relates to an autoinjector.

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

### Description of Related Art

Automatic injection devices are designed for automatic injection of a medical product into an injection site. Autoinjectors usually comprise a housing for receiving a medical container having a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to shield or unveil the needle and an injection mechanism for automatically injecting the medical into an injection site. The injection mechanism is usually triggered by a an activation component, also called a holder, when the needle is unveiled by the safety shield mechanism. It is important to prevent untimely triggering of the autoinjector, for example during transport or storage, and thus to prevent the activation component from triggering the injection mechanism unless the safety shield is properly retracted to the injection position by a user applying the autoinjector against the injection site. To that end, autoinjectors may, or may not, include a locker that prevent inadvertent move of the activation component.

There are three types of syringe flanges: the “cut flange” CF (FIG. 1A), the “round flange” and the “small round flange” RFS (FIG. 1B). The cut flange includes an anti-rolling feature in the form of two parallel edges a, b. The round flange and small round flange (FIG. 1B) have a circular shape. The small round flange has smaller dimensions than the cut flange or the round flange.

Autoinjectors are usually designed to cooperate with a cut flange. However, the cut flange may sometimes interfere with the safety shield mechanism because of its large diameter. In some cases, there is thus a need to use small round flanges, as illustrated in FIG. 1B, in autoinjectors. Small round flanges have a lower diameter than the cut flanges and thus avoid interference with the safety shield mechanism.

To assess the robustness of the autoinjectors, autoinjectors are subjected to drop tests as required in ISO11608. These drop tests usually consist in dropping the autoinjectors at least once from a height of 1 m onto a horizontal floor. There are three drop directions, as illustrated in FIGS. 2A to 2C: a drop ‘cap upward’ (FIG. 2A), a drop ‘cap downward’ (FIG. 2B), and a drop with the autoinjector being horizontal (FIG. 2C).

Medical containers are usually accommodated in a lower part of the housing. However, the medical containers may move a short distance in the proximal direction, i.e. inside the upper part of the housing, as it happens during a drop test ‘cap upward’ (FIG. 2A) or any accidental free fall ‘cap upward’ of the autoinjector onto the floor.

In order to stop this proximal movement of the medical container, the housing is usually provided with abutment protrusions that protrude from an inner wall of the upper part of the housing. These abutment protrusions are designed to abut against the cut flange or round flange of the medical container. This avoids that the medical container moves too far in the upper housing and accidentally triggers the injection mechanism.

However, a problem occurs when the medical container has a small round flange. Due to its reduced dimensions, the small round flange is no more capable of abutting against the abutment protrusions c of the housing (see FIGS. 3A-3B and 4A-4B). As a result, during a drop test ‘cap upward’, the medical container is free to move proximally inside the upper part of the housing. Generally, the medical container will abut against the activation component, or the locker if the autoinjector has a locker, thereby propelling the activation component, or the locker, in the proximal direction. The medical container, filled with the medical product, heavily weighs on said activation component or locker, and the accumulated kinetic energy of the filled medical container and activation component or locker may cause the activation component to reach a position that triggers the injection mechanism. That is, the autoinjector may be inadvertently activated.

It is known from the document WO2019011688 an administration assembly for a medicament delivery device and a medicament delivery device comprising this administration assembly.

There is therefore a need for an autoinjector that may be provided with a small round flange and that does not transition from a deactivated condition to activated condition when falling down on the floor during a drop test ‘cap upward’ or any accidental drop.

### SUMMARY OF THE INVENTION

An aspect of the invention is an autoinjector, for automatic injection of a product into an injection site, said autoinjector having a proximal end and a longitudinal axis A, the autoinjector comprising:
a housing configured to receive a medical container, said medical container having a barrel defining a reservoir for containing a medical product, and said barrel having a distal end provided with a needle and an opened proximal end configured to receive a plunger rod for pushing a stopper arranged inside the barrel,
a needle cover coupled to and movable with respect to said housing between a first extended position, wherein the needle cover at least partially shields the needle, a retracted position, wherein the needle cover moves proximally inside the housing to unshield the needle, and a second extended position wherein the needle cover moves back in the distal direction to shield the needle,
an injection mechanism configured to move the plunger rod distally inside the barrel in order to expel the medical product contained inside the barrel,
a holder movable with respect to the housing between a passive position wherein the holder does not trigger the injection mechanism and an active position wherein the holder triggers the injection mechanism,
blocking means configured to prevent the medical container from moving proximally inside the housing, such that the medical container cannot displace the holder towards the active position if the autoinjector falls down onto a floor, proximal end first.

As a result, the autoinjector remains in a deactivated condition.

The holder is axially movable along the longitudinal axis A between the passive and the active position. The movement of the holder from the passive to the active position is caused by the needle cover abutting against the holder when the needle cover moves towards the retracted position.

The blocking means are configured to block any proximal movement of the medical container in the housing. This results in the medical container being prevented to move in the proximal direction when the autoinjector hits a floor cap upward, and in any other situation where the medical container would otherwise have moved in the proximal direction (for instance a user shaking the autoinjector).

In an embodiment, the blocking means include a friction-fit ring which is secured to a locking element configured to lock the needle cover in the second extended position, and the friction-fit ring defines a central opening for frictionally engaging the barrel such that proximal movement of the barrel is prevented.

The friction-fit ring is arranged on the locking element such that the needle cover does not interact with the friction-fit ring. The friction-fit ring may be secured to a proximal portion, which may be in the form of a ring portion, of the locking element whereas an opposite distal portion of the locking element, such as proximally extending legs, engages the needle cover to block the needle cover in the second extended position (safety position). Besides, an outer diameter of the friction-fit ring may be lower than an outer diameter defined by a ring portion of the locking element.

The friction-fit ring has a central opening coaxial to a central opening of the locking locking element.

The locking element is fixed relative to the housing.

In an embodiment, the friction-fit ring includes radial protrusions protruding from an inner wall of the central opening, said radial protrusions defining an inner diameter lower than an outer diameter of the barrel, and said radial protrusions longitudinally extending in a circumferential direction.

In an embodiment, the radial protrusions have a slanted proximal wall for allowing insertion of the barrel in the distal direction through the central opening.

In an embodiment, the radial protrusions are made of a resilient material.

In an embodiment, the friction-fit ring is configured to define an accommodation cavity between an inner wall of the central opening and the barrel, said accommodation cavity being configured to receive a glue material.

In an embodiment, the friction-fit ring includes snap-fitting means allowing a snap-fit connection with a flange of the barrel.

In an embodiment, the friction-fit ring is overmolded on the locking element.

In an embodiment, the blocking means include a spacer ring defining a central opening for allowing insertion of the plunger rod, the spacer ring being configured to abut on one side against a flange of the medical container, and the spacer ring having radially protruding lugs so as to abut, on the other side, against an abutment surface of the housing.

In an embodiment, the lugs have an increasing width in an outward direction.

In an embodiment, the abutment surface of the housing is defined by an axial rib of the housing.

In an embodiment, the spacer ring has snap-fitting means for removable attachment to the holder.

In an embodiment, the snap-fitting means include two, and preferably only two, axial protrusions that are diametrically offset.

In an embodiment, the spacer ring has a recessed edge portion for receiving a portion of a locker, said locker being coupled to the holder, and the locker being movable with regard to the housing between a locking position, wherein the locker prevents the holder from moving to the activated position, and an unlocking position, wherein the locker rotates around the longitudinal axis A for allowing the holder to move to the activated position, rotation of the locker from the locking to the unlocking position being caused by the needle cover moving towards the retracted position.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIGS. 1A and 1B are perspective views of, respectively, a so-called cut flange and a so-called small round flange,

FIGS. 2A-2C are perspective views of, respectively, a first vertical drop position (cap upward), a second vertical drop position (cap downward), and a third drop position (horizontal),

FIGS. 3A and 3B are perspective views of autoinjectors provided with, respectively, a syringe cut flange and a syringe small round flange,

FIGS. 4A and 4B are cross-section views of autoinjectors provided with, respectively, a syringe cut flange and a syringe small round flange, in a plane orthogonal to a longitudinal axis A,

FIGS. 5A and 5B are are cross-section views of autoinjectors provided with, respectively, a syringe cut flange and a syringe small round flange, in a longitudinal plane including the longitudinal axis A,

FIG. 6 is a partial cross-section view of an autoinjector according to an embodiment of the invention,

FIG. 7 is a perspective view of the lower part and the upper part of an autoinjector according to an embodiment of the invention,

FIG. 8 is an exploded view of an autoinjector according to an embodiment of the invention,

FIG. 9 is a perspective view of a holder and a locker of an autoinjector according to an embodiment of the invention,

FIG. 10 is a perspective view of a locker of an autoinjector according to an embodiment of the invention,

FIG. 11 is a cross-section view in perspective of an upper housing of an autoinjector according to an embodiment of the invention,

FIG. 12A is a perspective view of an autoinjector according to an embodiment of the invention, wherein the upper housing is removed,

FIG. 12B is a detail of FIG. 12A,

FIG. 12C is cross-section view in a longitudinal plane of an autoinjector according an embodiment of the invention,

FIG. 12D is a perspective view of a friction-fit ring of an autoinjector according an embodiment of the invention,

FIG. 12E is a top view of a friction-fit ring of an autoinjector according an embodiment of the invention,

FIG. 13A is a perspective view of an autoinjector according to an embodiment of the invention, wherein the upper housing is removed,

FIG. 13B is a detail of FIG. 13A,

FIG. 13C is a perspective view of a friction-fit ring of an autoinjector according an embodiment of the invention,

FIG. 13D is a top view of a friction-fit ring of an autoinjector according an embodiment of the invention,

FIG. 14A a perspective view of an autoinjector according to an embodiment of the invention, wherein the upper and lower housings are removed,

FIGS. 14B and 14C are details of FIG. 14A,

FIG. 14D is a perspective view of a spacer ring of an autoinjector according an embodiment of the invention,

FIG. 14E is a top view of a spacer ring of an autoinjector according an embodiment of the invention,

FIG. 14F is a partial cross-section view of an autoinjector according an embodiment of the invention,

FIG. 14G is a cross-section view in a longitudinal plane of an autoinjector according an embodiment of the invention,

FIG. 14H is a perspective view of the distal end of a holder of an autoinjector according to an embodiment of the invention,

FIGS. 15-27 are perspective views illustrating operating steps of an autoinjector according to an embodiment of the invention.

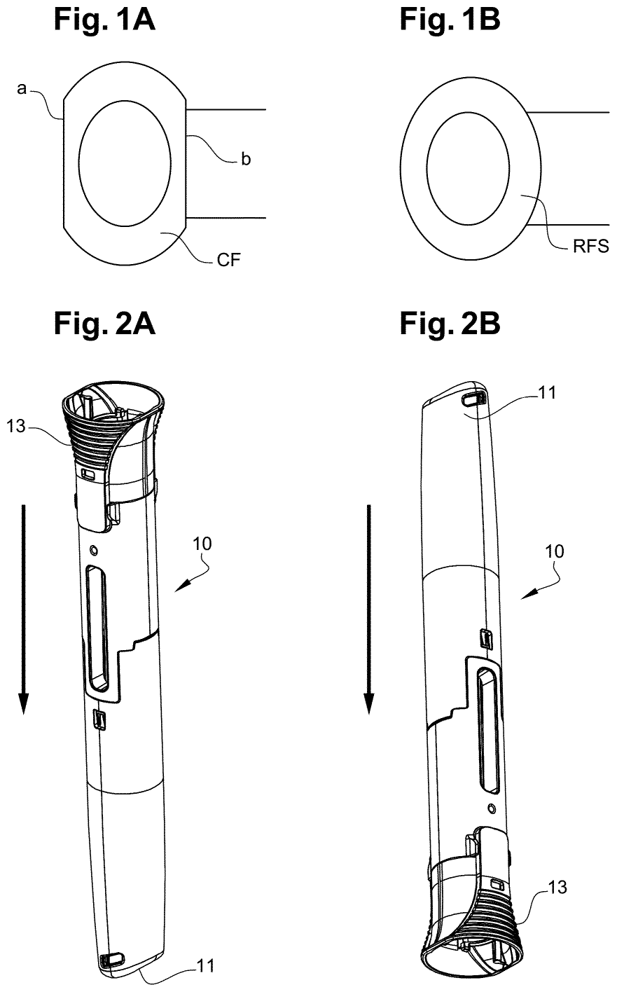

### DESCRIPTION OF THE INVENTION

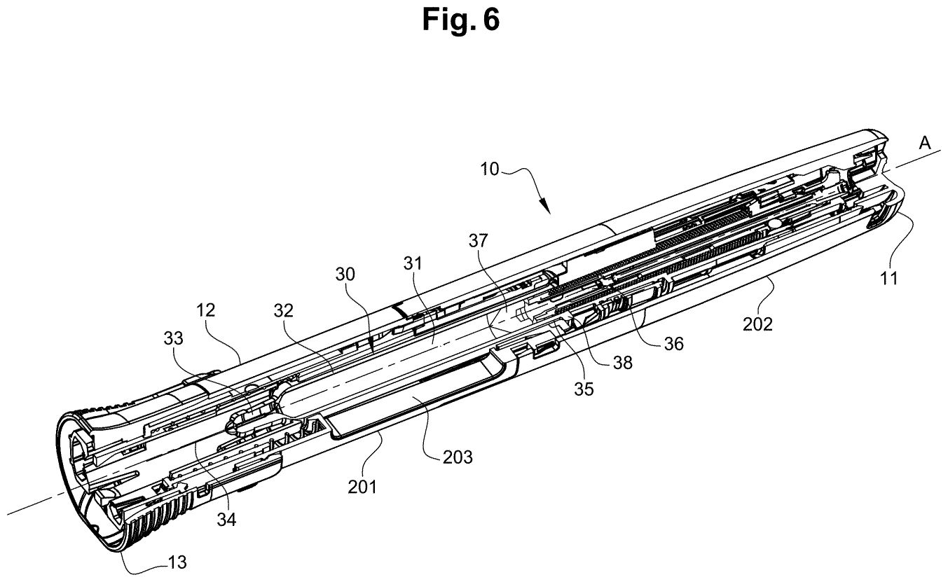

With reference to FIGS. 6 and 7 is shown an autoinjector 10 according to an embodiment of the invention. The autoinjector 10 is designed for automatic injection of a product 31 into an injection site. The autoinjector 10 comprises a housing 20 extending along a longitudinal axis A. The housing 20 is formed by the assembly of a lower housing 201 and an upper housing 202. The lower housing 201 and the upper housing 202 may be removably secured one to another by securing means such as, for instance, snap-fitting means. The autoinjector 10 has a proximal end 11 and a distal end 12. A cap 13, including a retainer 14, is removably attached to the distal end 12.

The lower housing 201 is configured to receive a medical container 30, such as a prefilled syringe. The medical container 30 has a barrel 32 defining a reservoir for containing the product 31. The lower housing 201 may define an inspection window 203 for allowing a user to visually inspect the product 31 contained within the reservoir of the medical container 30.

The barrel 32 has a distal end 33 provided with a needle 34 and an opened proximal end 35 for receiving a plunger rod 36. The plunger rod 36 may have a threaded or, preferably, a non-threaded distal end. The plunger rod 36 is configured to distally push a stopper 37 arranged inside the barrel 32 so as to expel the product 31 via the distal end and the needle 34. The opened proximal end 35 of the barrel 32 includes a small round flange 38 as illustrated in FIG. 6.

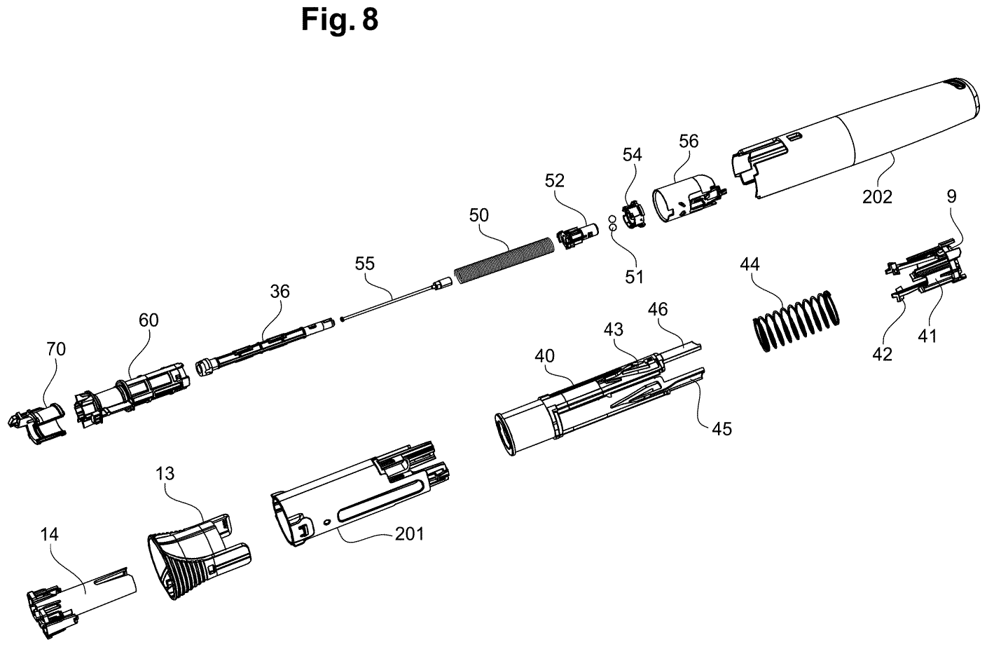

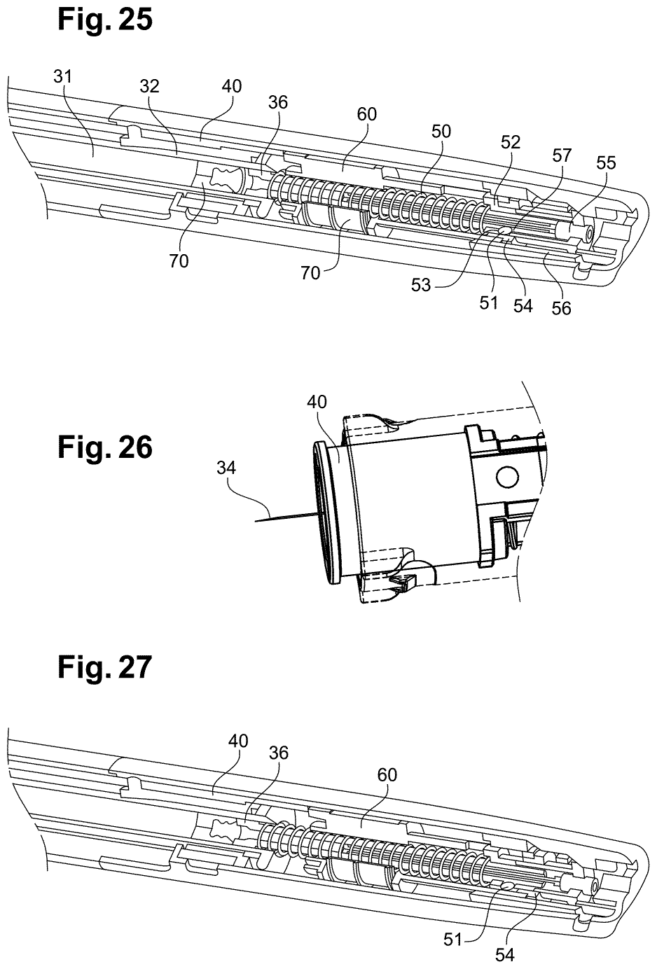

With reference to FIG. 8, the autoinjector 10 comprises a safety shield mechanism including a needle cover 40. The needle cover 40 is coupled to and movable with respect to the lower housing 201 between a first extended position (pre-use position), wherein the needle cover 40 at least partially shields the needle 34, a retracted position (injection position) wherein the needle cover 40 moves proximally inside the lower housing 201 to unshield the needle 34 (FIG. 25), thereby allowing insertion of the needle 34 into the injection site, and a second extended position (safety position), wherein the needle cover 40 moves back in the proximal direction so as to safely shield the needle 34. The safety shield mechanism includes locking means for locking the needle cover 40 in the second extended position (safety position), thereby preventing needle stick injuries. The locking means may include a locking element 41 having a proximally extending resilient leg 42 that engages a two-way slot 43 arranged through the needle cover 40. The safety shield mechanism further includes return means, such as a safety spring 44, configured to move the needle cover 40 from the retracted position to the second extended position after injection of the medical product 31 into the injection site. The needle cover 40 has a first and a second proximally extending legs 45, 46 whose function will be hereinafter described in further details.

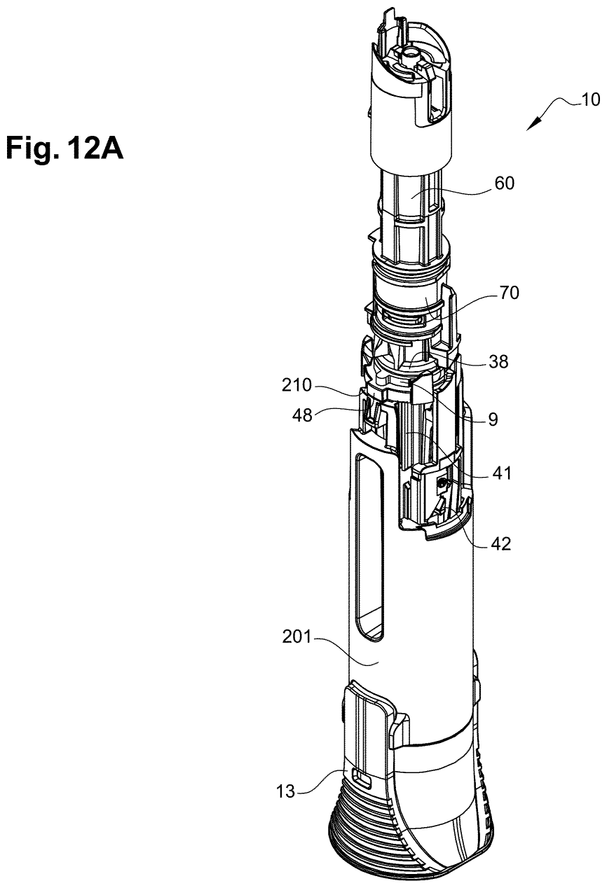

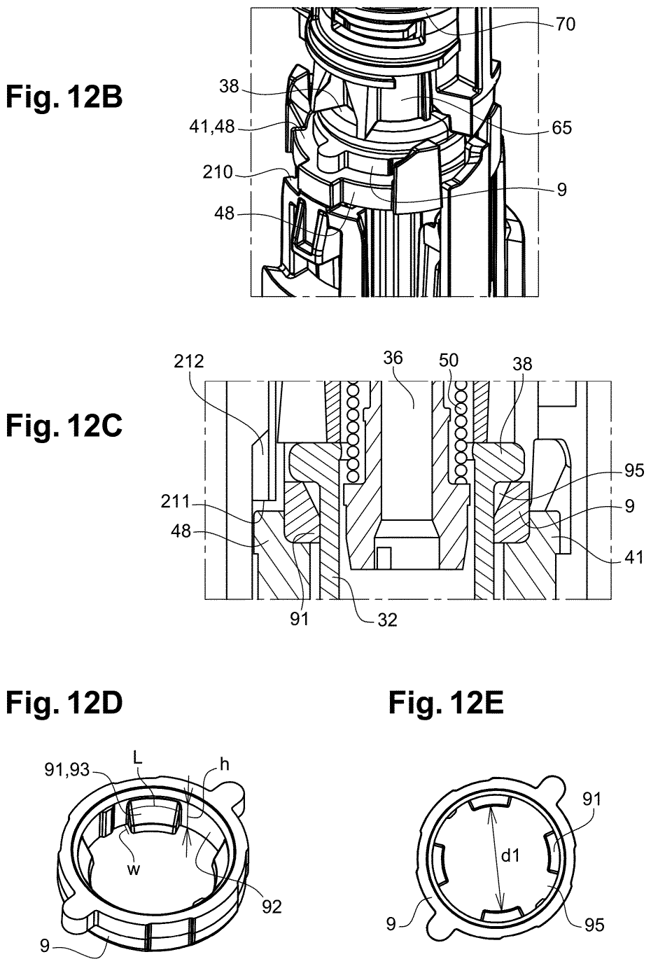

With reference to FIGS. 12B and 12C, the locking element 41 has a ring portion 48 defining a central opening for accommodating the barrel 32 of the medical container. The locking element 41 is blocked in the distal direction by its ring portion 48 abutting against a proximal leg 210 of the lower housing 201, and blocked in the proximal direction by its ring portion 48 abutting against a blocking surface 211 of the upper housing 202, so that the locking element 41 is substantially fixed relative to the housing 20. As illustrated in FIG. 12C, the blocking surface 211 of the upper housing 202 may be defined at a distal end of one or several axial blocking ribs 212.

The autoinjector 10 has an injection mechanism configured to automatically perform injection of the product 31 into the injection site. The injection mechanism includes an injection spring 50 for pushing the plunger rod 36 in the distal direction, locking balls 51 radially movable from a locking position wherein they block distal movement of the plunger rod 36 to an unlocking position wherein the locking balls 51 allow the plunger rod 36 to move in the distal direction under the action of the injection spring 50, thereby performing the injection. The injection mechanism has a washer 52 defining a radial cavity 53 for accommodating the locking balls 51, and a ring 54 coupled to and movable with respect to the washer 52 between a first position wherein the ring 54 maintains the locking balls 51 inside a recess 57 of the plunger rod 36 and a second position wherein the ring 54 allows the locking balls 51 to leave the plunger rod recess 57. The autoinjector 10 may further include a centerpiece 55 and an indicator 56 for providing the user with an audible, visual or tactile feedback that the injection is completed.

The autoinjector 10 has a holder 60 configured to trigger the injection mechanism. The holder 60 is movable within the upper housing 202 from a passive to an active position. In the passive position, the holder 60 is away from the ring 54 and thus does not trigger the injection. In the activated position, the holder 60 moves the ring 54 from the first to the second position. The proximal movement of the holder 60 from the passive to the activated position is caused by the second proximal leg 46 of the needle cover 40 abutting against the holder 60 when the needle cover 40 moves from the first extended position (pre-use position) towards the retracted position (injection position).

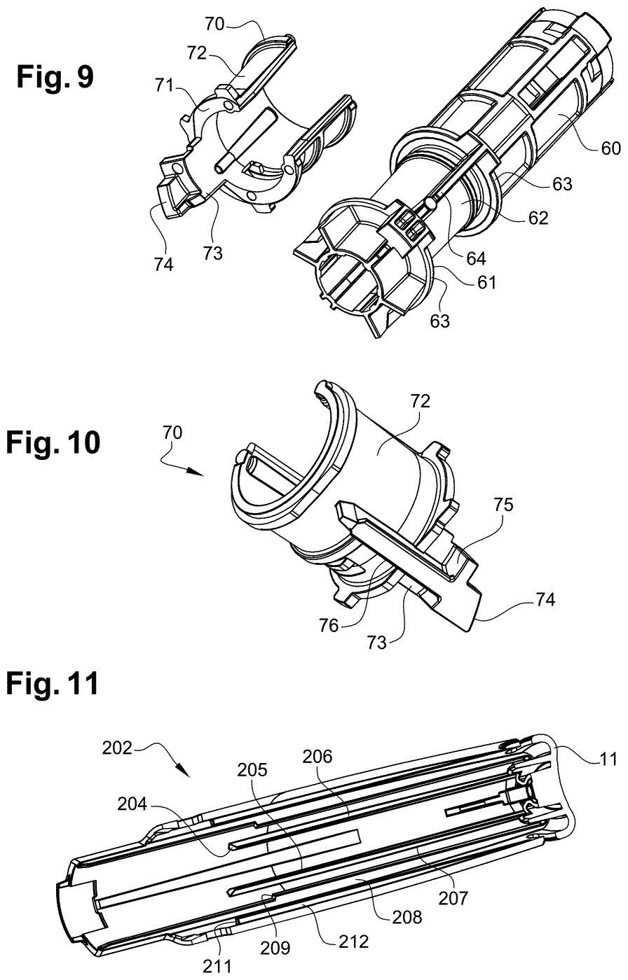

The autoinjector 10 preferably includes a locker 70, in the form of a C-shaped ring, as illustrated in FIGS. 9-10. The locker 70 is configured to prevent inadvertent actuation of the autoinjector 10 and, more specifically, to block any accidental movement of the holder 60 towards the activated position. To that end, the locker 70 is coupled to and movable with respect to the holder 60. The locker 70 has a distal abutment surface 71 for abutting against a proximal shoulder 61 of the holder 60. The locker 70 may slide and rotate around the longitudinal axis A with respect to the holder 60 and the upper housing 202. The holder 60 has a groove 62 for accommodating the locker 70. The groove 62 may be defined by two circumferential ribs 63 that limit the axial movement of the locker 70 relative to the holder 60. The proximal shoulder 61 of the holder 60 may be delimited by one of these circumferential ribs 63. The groove 62 may further include an axial rib 64 that blocks the rotational movement of the locker 70 around the holder 60.

The locker 70 has a ring portion 72, that slides in the groove 62 of the holder 60, and a distal leg 73, that distally protrudes from the ring portion 72. The distal leg 73 has a cam portion 74 (at a distal end thereof), a proximal abutment surface 75 and a lateral abutment surface 76. With reference to FIG. 11, the upper housing 202 may have one or several first axial ribs 206, one or several second axial ribs 207, and one or several third axial ribs 208, extending along the longitudinal axis A and protruding from an inner lateral wall of the upper housing 202. The first axial rib 206 defines the first abutment surface 204, which may be arranged at a distal end of this first axial rib 206. The first abutment surface 204 is configured to stop a proximal movement of the locker 70, by abutting against the proximal abutment surface 75 of the locker 70, and thus temporarily block the locker 70 in an intermediate blocking position. The second axial rib 207 has a second abutment surface 205, which may be arranged at one side of the second axial rib 207, configured to stop rotation of the locker 70, by abutting against the lateral abutment surface 76 of the locker 70, thereby positioning the locker 70 in a release position. The third axial rib 208 defines a third abutment surface 209, which may be arranged on a distal shoulder of the third axial rib 208.

The locker 70 is movable between an initial position wherein the locker 70 is away from a first abutment surface 204 of the upper housing 202, an intermediate blocking position wherein the locker 70 moves proximally and abuts against the first abutment surface 204 of the upper housing 202, and a release position wherein the locker 70 rotates away from a first axial rib 206 and abuts against a second axial rib 207 of the upper housing 202. In the intermediate blocking position, the locker 70 prevents the holder 60 from being moved towards the activated position. In the release position (i.e. after rotation), the locker 70 is free to move proximally and accordingly allows the holder 60 to move proximally towards the activated position. In the release position of the locker 70, the autoinjector 10 is ready for activation. The autoinjector 10 is activated (the injection mechanism is triggered) when the holder 60 reaches the activated position.

As above-mentioned, the movement of the locker 70 from the initial to the intermediate blocking position, and then to the release position, i.e. the normal activation of the autoinjector 10, is caused by the needle cover 40 abutting against the cam portion 74 of the locker 70 while the needle cover 40 moves from the first extended position to the retracted position.

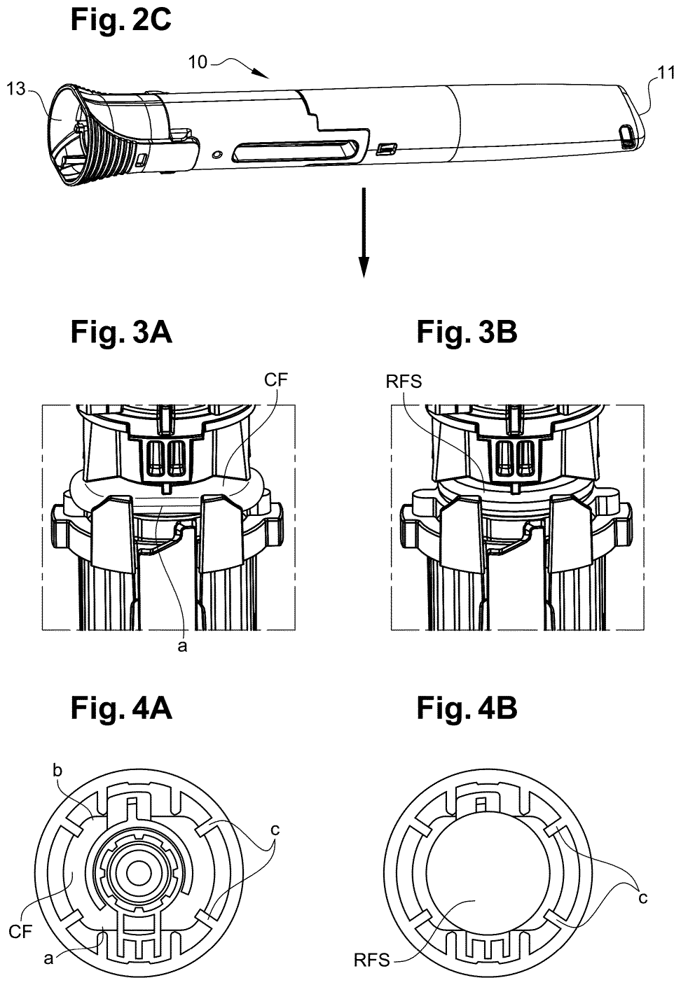

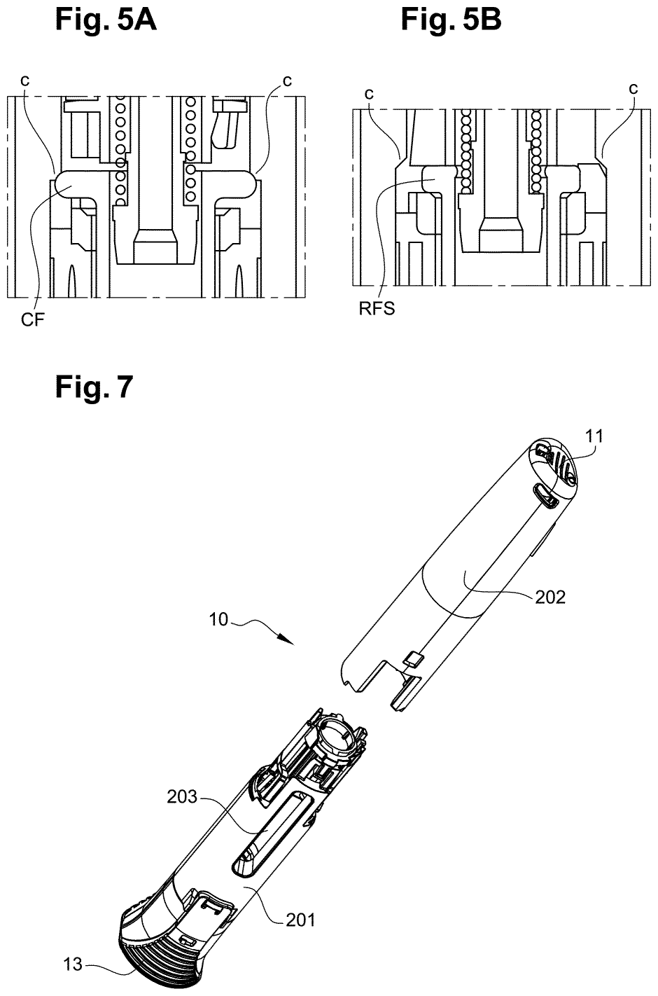

In order to prevent inadvertent transition of the autoinjector 10 from the deactivated condition to the activated condition during a drop test ‘cap upward’ or ‘proximal end first’ (as illustrated in FIG. 2A) wherein the medical container 30 having a small round flange 38 may move in the proximal direction and thus heavily weighs on the holder 60 or on the locker 70, the autoinjector 10 comprises blocking means configured to stop the medical container 30 and prevent the medical container 30 from causing displacement of the holder 60 towards the active position.

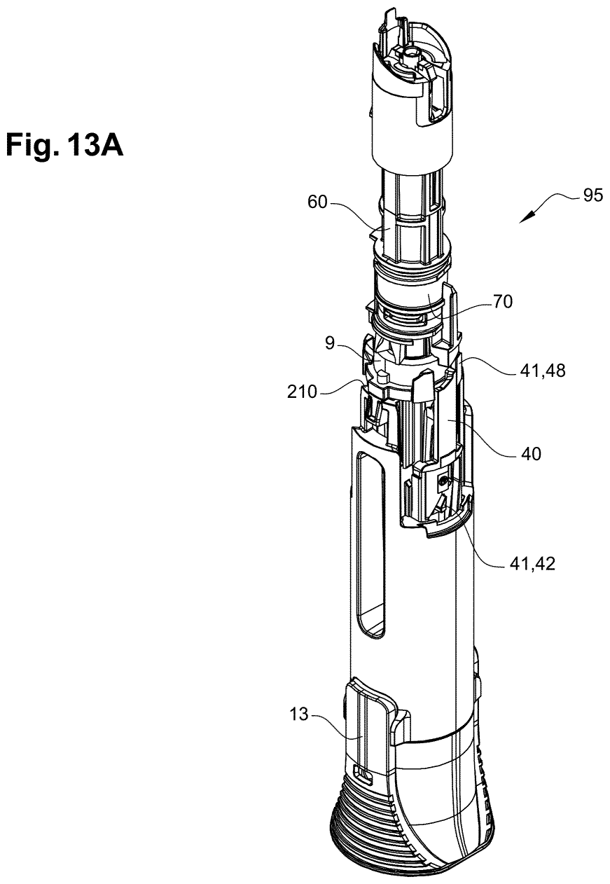

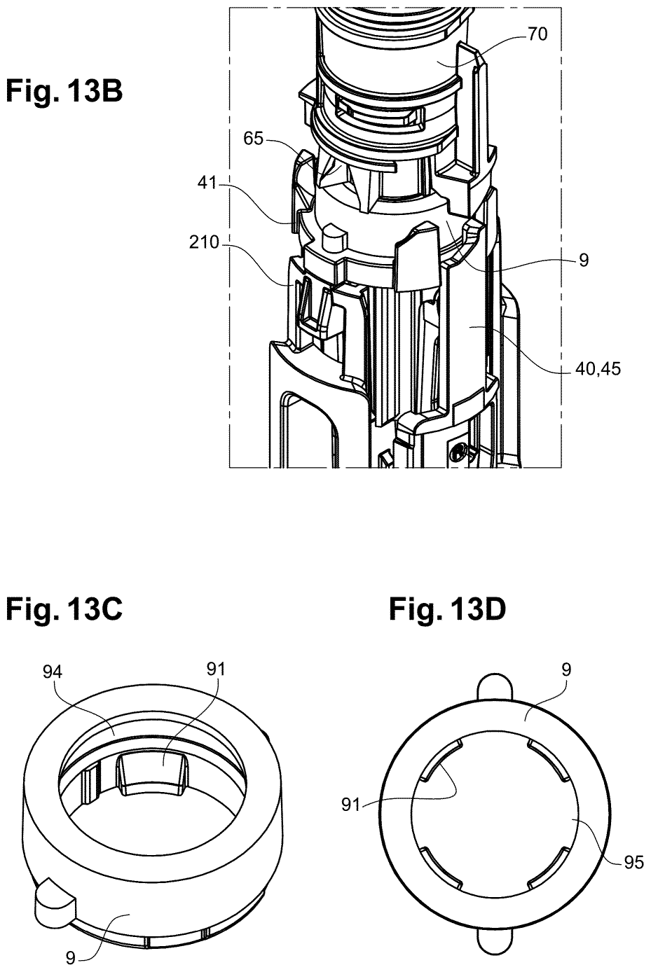

According to an embodiment illustrated in FIGS. 12A-12E and 13A-13D, the blocking means include a friction-fit element, which may be in the form of a friction-fit ring 9, and which is secured to the locking element 41. For example, the friction-fit ring 9 may be overmolded with the locking element 41. The friction-fit element is configured for allowing a friction-fit engagement with the barrel 32 so that proximal movement of the medical container 30 is prevented.

The friction-fit ring 9 defines a central opening for frictionally engaging the barrel 32. Due to the friction interference between the friction-fit ring 9, which may be made of a resilient material such as thermoplastic elastomer (TPE), and the glass barrel 32, the medical container 30 is blocked in the proximal direction and cannot cause inadvertent activation of the autoinjector 1. As illustrated in FIGS. 12A-12C, the friction-fit ring 9 may be arranged at the proximal end of the barrel 32, between the ring portion 48 of the locking element 41 of the safety shield mechanism and the flange 38 of the medical container 30.

With reference to FIGS. 12D and 12E, the friction-fit ring 9 may include radial protrusions 91 inwardly protruding from an inner wall 92 of the central opening. For instance, the friction-ring 9 has four radial protrusions 91 regularly distributed in a circumferential direction. In order to create interference with the barrel 32, the central opening of the friction-fit defines an inner diameter lower than an outer diameter of the barrel 32. More specifically, the diameter d1 defined by the innermost portions of the radial protrusions 91 is lower than the outer diameter of the glass barrel 32. FIG. 12C illustrates the resulting interference. Still with reference to FIGS. 12D and 12E, the protrusions 91 may longitudinally extend in a circumferential direction in order to improve their blocking action. That is, their length L is the greatest dimension of the protrusions 91, in comparison with their height h or width w. It is contemplated that the protrusions 91 may have a slanted proximal wall 93 for allowing insertion of the barrel 32 in the distal direction through the central opening of the friction-fit ring 9. The protrusions 91 may be made of a resilient material such as a thermoplastic elastomer (TPE) to deform outwardly radially when the barrel 32 is inserted and to exert on the barrel an inward radial force that help blocking the medical container 30 should the autoinjector 10 falls down on a floor.

According to the embodiment illustrated in FIGS. 13A-13D, the friction-fit ring 9 includes snap-fitting means configured to allow a snap-fit engagement between the medical container 30 and the friction-fit ring 9. This helps secure the medical container 30 to the friction-fit ring 9. The snap-fitting means may comprise a circumferential groove 94 extending through the inner wall 92 and configured to engage the flange 38 of the barrel 32. The circumferential groove 94 may be distally located with to the protrusions 91, in a distal portion of the friction-fit ring 9 while the protrusions 91 are located in a proximal portion of the friction-fit ring 9. It is important that the friction-fit ring 9 be made of a resilient material so that the distal portion of the friction-fit ring 9 may deform in order to receive the flange 38.

It is contemplated that the friction-fit ring 9 may define one or several accommodation cavities 94, between the inner wall 92 and the barrel 32, and these accommodation cavities may be filled with a glue material so as to secure the medical container 30 to the friction-fit ring 9 and the locking element 41.

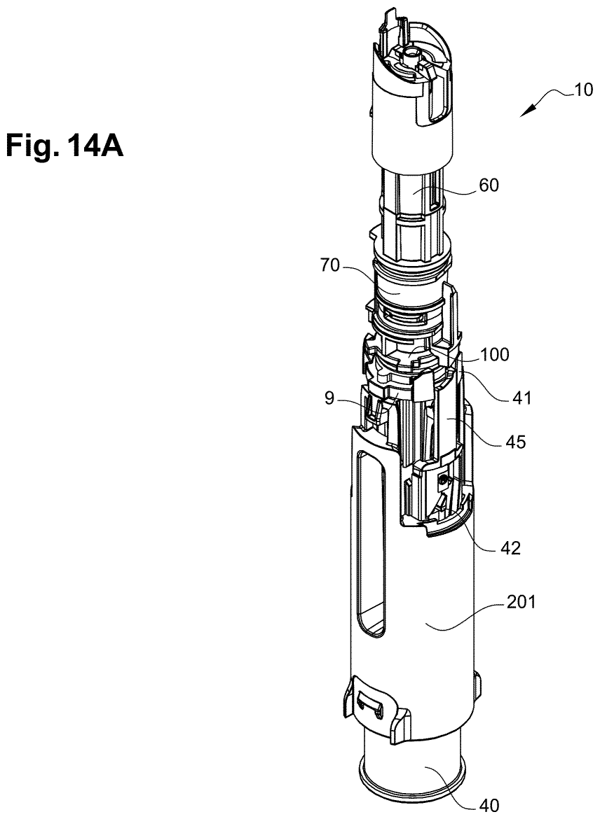

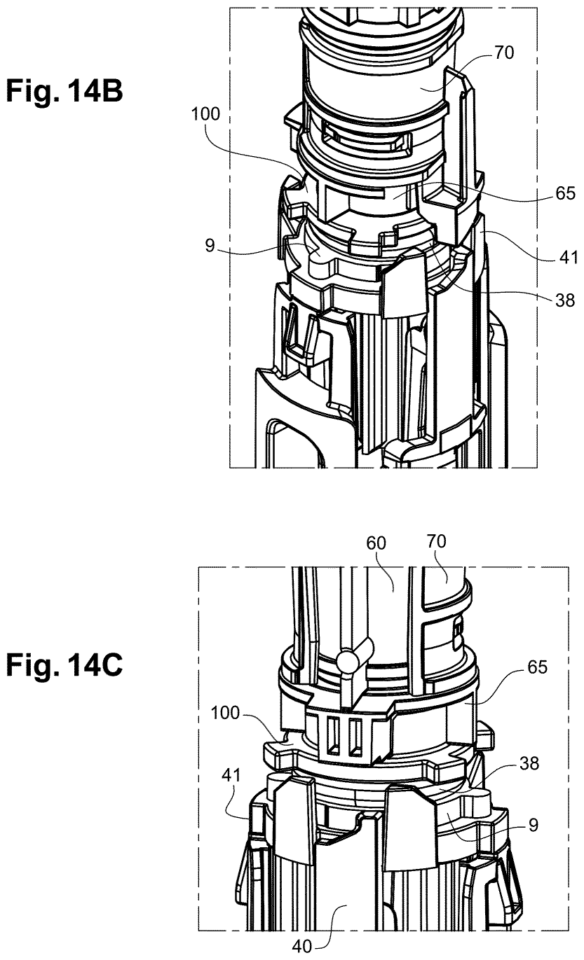

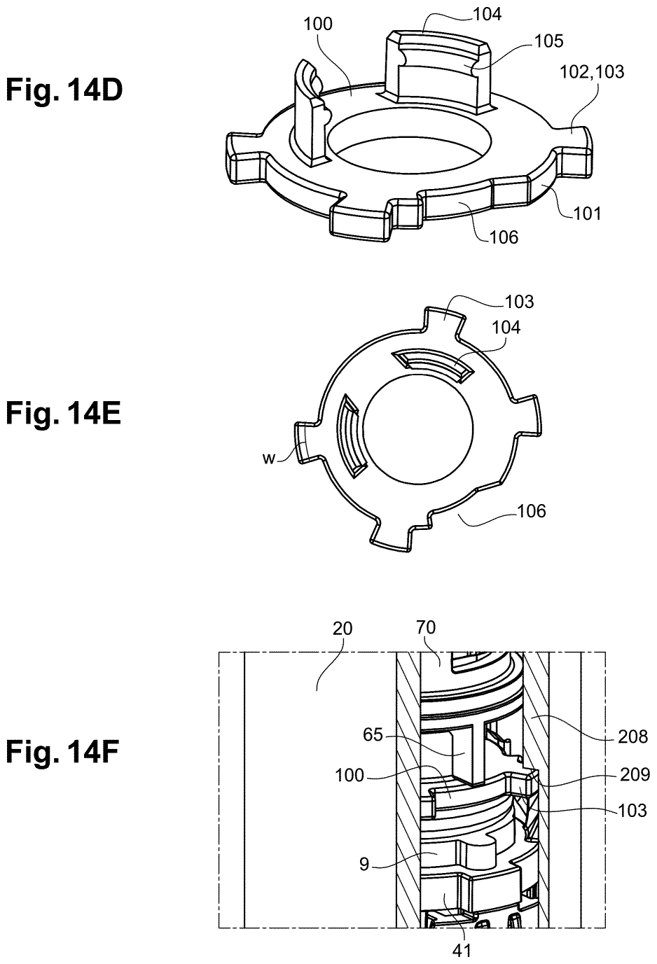

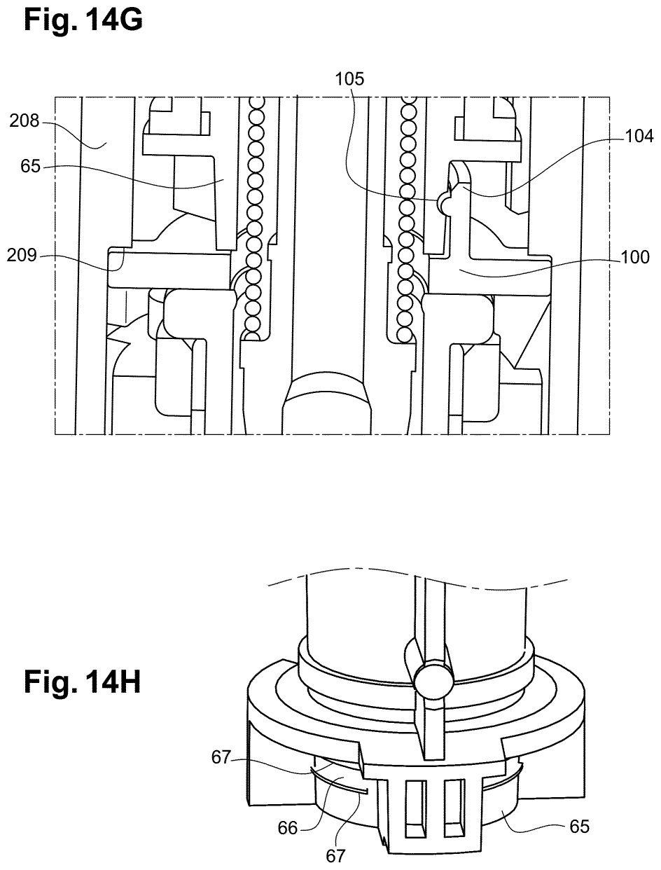

In an embodiment illustrated in FIGS. 14A to 14H, the blocking means include a spacer, which may be in the form of a spacer ring 100 defining a central opening for allowing insertion of the plunger rod 36. The spacer 100 is arranged between the flange 38 of the medical container 30 and the distal end 65 of the holder 60. The spacer 100 includes a larger diameter than the small rounded flange 38. More specifically, the spacer 100 has a distal abutment surface 101 abutting against the flange 38 of the medical container 30, and a proximal abutment surface 102 configured to abut against the third abutment surface 209 of the upper housing 202 (see FIGS. 14F and 14G). Therefore, the spacer 100 blocks a proximal movement of the medical container 30 if the autoinjector 10 hits the floor ‘cap upward’. The holder 60 accordingly does not transition to the active position and the autoinjector 10 remains deactivated.

The proximal abutment surface 102 of the spacer ring 100 may be defined by radially protruding lugs 103, each of said lugs 103 abutting against a different one of the third axial ribs 208 of the upper housing 202. For example, the spacer ring 100 may include two pairs of diametrically opposite lugs 103. With reference to FIG. 14E, the lugs 103 have an increasing width w in an outward direction. For instance, they may have a trapezoid shape.

As illustrated in FIGS. 14D, 14E and 14G, the spacer ring 100 advantageously has snap-fitting means for removable attachment to the holder 60. This permits temporary attachment of the spacer ring 100 to the upper housing 202 so that the final assembly of the autoinjector 10 remains substantially unchanged: the lower and upper housings 201, 202 just need to be secured one to each other in a known way. The spacer ring 100 automatically gets into position against the syringe flange 38.

It is however important that the spacer ring 100 be able to disengage the holder 60 during normal use of the autoinjector 1, so that the holder 60 may move to the active position during an injection operation. Accordingly, the snap-fitting means may include two axial protrusions 104 that are not regularly distributed in the circumferential direction. That is, the two axial protrusions 104 may not face each other and thus be diametrically offset. Preferably, they are located of a same half of the spacer ring 100. Also, there may be only two axial protrusions 104. This limits the attachment of the spacer ring 100 to the holder 60 and allows the holder 60 to easily disengage the spacer ring 100 when being moved towards the active position. The snap-fitting means may include a circumferential rib 105, extending for example on an inner wall of the axial protrusions 104, and a recess 66 that may be delimited by two ribs 67 arranged on the distal end 65 of the holder 60 (see FIG. 14H).

As best shown in FIG. 14E, the spacer ring 100 may have a recessed edge portion 106 for receiving the distal leg 73 of the locker 70. Thus, the spacer ring 100 does not hinder rotation of the locker 70 between the locking position and the unlocking position.

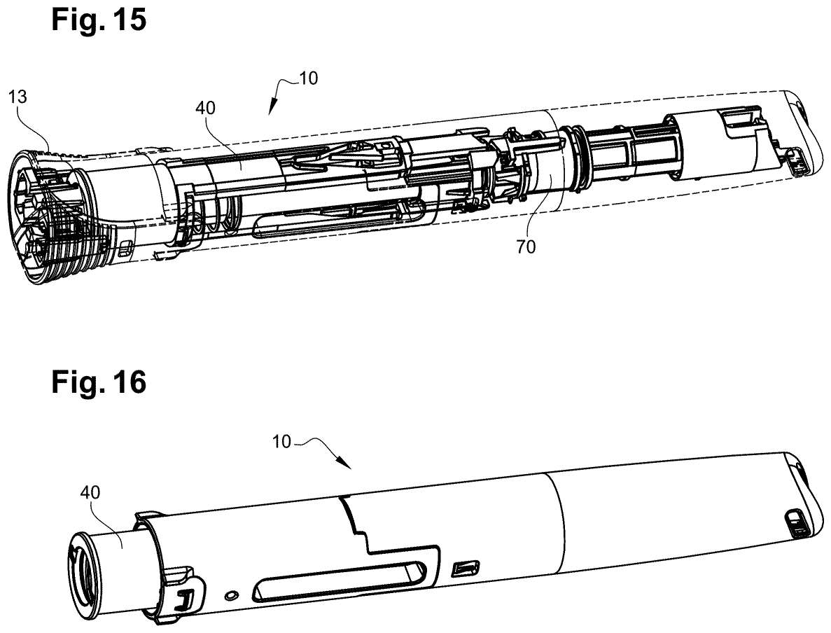

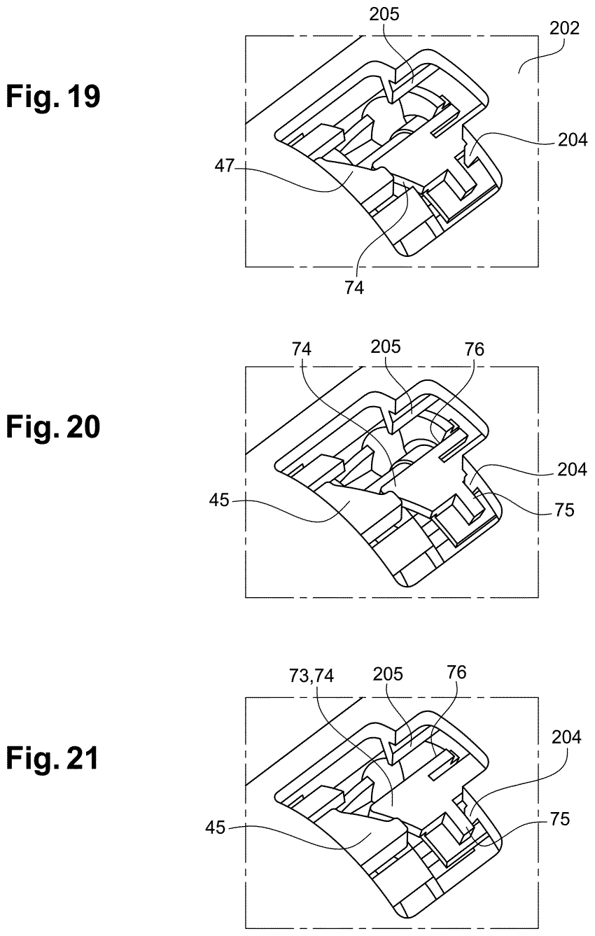

The normal operation of the autoinjector 10 will now be described hereafter with reference to the FIGS. 15-25.

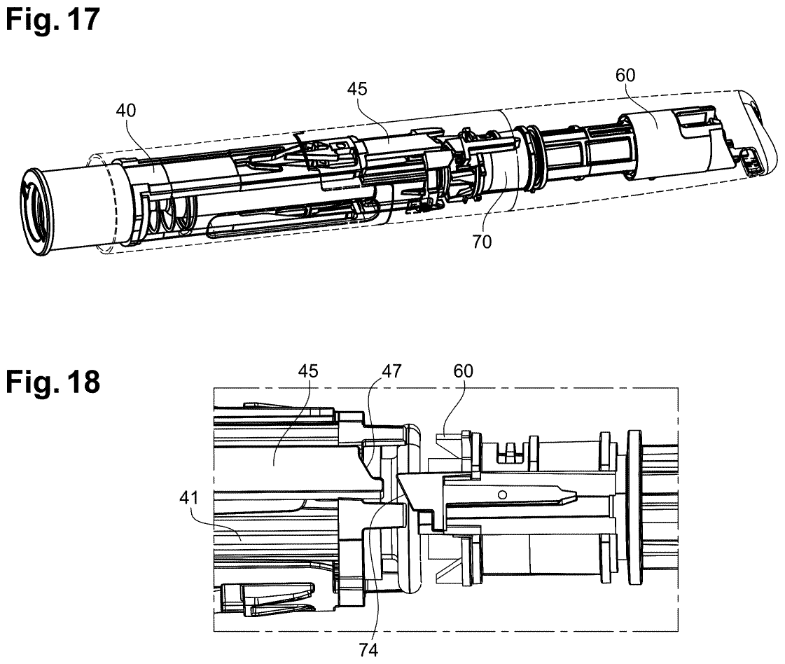

The user firstly removes the cap 13 (FIGS. 16-17). The needle cover 40 is in the first extended position (pre-use position) and therefore conceals the needle 34. The locker 70 is in the initial position and the holder 60 is in the deactivated position.

The user then applies a distal end of the needle cover 40 against the injection site and pushes the autoinjector 10 against the injection site. This causes the needle cover 40 to move in the proximal direction towards the retracted position. By doing so, the first proximal leg 45 of the needle cover 40 comes in abutment against the cam portion 74 of the locker 70 (FIGS. 17-18).

Further movement of the needle cover 40 pushes the locker 70 in the proximal direction. The locker 70 slides along the holder 60 and the housing 20 until the proximal abutment surface of the locker 70 abuts against the first abutment surface 204 of the upper housing 202 (FIG. 20). The locker 70 is in the intermediate blocking position.

A further movement of the needle cover 40 is required to make the autoinjector 10 transitions from the deactivated to an intermediate condition.

Due to the shape of the locker cam portion 74 and the needle cover cam portion 47, and due to the abutment between the locker 70 and the upper housing 202, further movement of the needle cover 40 in the proximal direction causes the locker 70 to rotate around the holder 60 (FIG. 21).

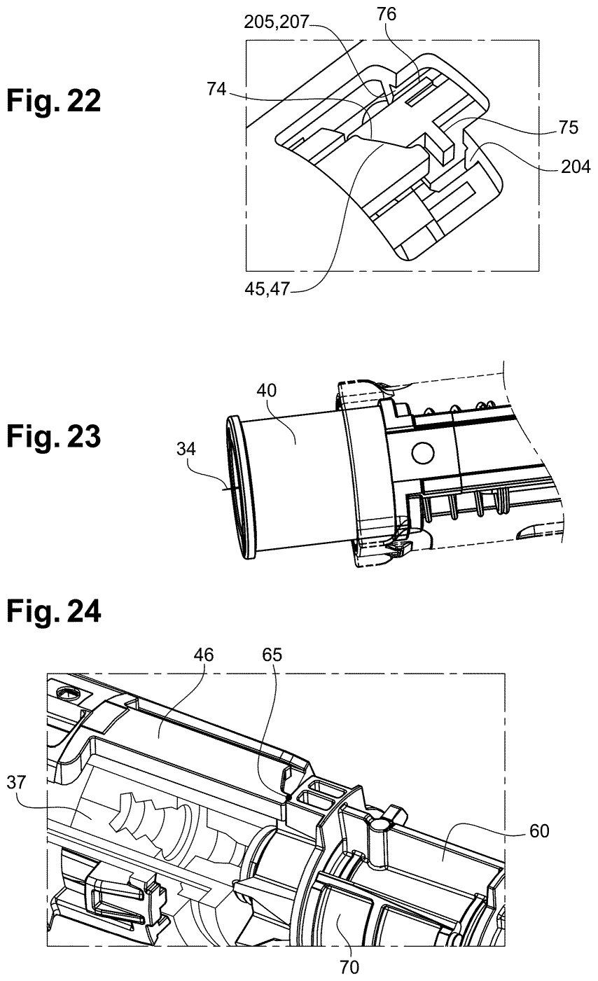

The locker 70 rotates until the lateral abutment surface comes against the second abutment surface 205 of the upper housing 202 (FIG. 22). The locker 70 is in the release position. The autoinjector 10 is ready for being activated.

At that time, the needle cover 40 may unveil the needle 34 and the needle 34 may begin insertion into the injection site (FIG. 23).

The second proximal leg 46 of the needle cover 40 comes against a distal end 65 of the holder 60 (FIG. 23) and pushes the holder 60 in the proximal direction (FIG. 24). The holder 60 leaves the passive position and progresses towards the activated position.

At this stage, the locking balls 51 extend in the locking recess of the plunger rod 36 and thus prevent the injection spring 50 from pushing the plunger rod 36 in the distal direction. The locking ring is in the first position such that the locking ring blocks the locking balls 51 inside the lokcing recess of the plunger rod 36.

However, when the needle cover 40 reaches the retracted position (injection position), as illustrated in FIGS. 26 and 27, the holder 60 reaches the activated position. The holder 60 comes in abutment against the locking ring and moves the locking ring in the proximal direction to the second position. The locking balls 51 are hence free to move radially outwardly, outside the locking recess of the plunger rod 36. As a result, the plunger rod 36 is no more maintained by the locking balls 51 and the injection spring 50 extends, moving the plunger rod 36 in the distal direction. The plunger rod 36 then pushes the stopper 37 inside the barrel 32, thereby expelling the product 31 through the needle 34 into the injection site. Injection is triggered, and the autoinjector 10 is activated.

At the end of the injection, the centerpiece 55 cooperates with the plunger rod 36 and the indicator to provide the user with a feedback indicating that the injection is completed.

The user may take the autoinjector 10 away from the injection site. The safety spring 44 accordingly moves the needle cover 40 back in the distal direction, until the needle cover 40 reaches the second extended position (safety position) wherein the needle cover 40 shields the needle 34 and is prevented from moving back in the retracted position by the locking element 41.

By blocking the medical container 30 inside the lower housing 201, the invention permits to prevent inadvertent activation of the autoinjector 10 during a drop test ‘proximal end first’. The medical container 30, even if equipped with a small round flange 38, indeed cannot cause anymore the holder 60 to move towards the active position.

1. An autoinjector, for automatic injection of a product into an injection site, said autoinjector having a proximal end and a longitudinal axis A, the autoinjector comprising:
a housing configured to receive a medical container, said medical container having a barrel defining a reservoir for containing a medical product, and said barrel having a distal end provided with a needle and an opened proximal end configured to receive a plunger rod for pushing a stopper arranged inside the barrel,
a needle cover coupled to and movable with respect to said housing between a first extended position, wherein the needle cover at least partially shields the needle, a retracted position, wherein the needle cover moves proximally inside the housing to unshield the needle, and a second extended position wherein the needle cover moves back in the distal direction to shield the needle,
an injection mechanism configured to move the plunger rod distally inside the barrel in order to expel the medical product contained inside the barrel,
a holder movable with respect to the housing between a passive position wherein the holder does not trigger the injection mechanism and an active position wherein the holder triggers the injection mechanism, proximal movement of the holder from the passive to the active position being caused by the needle cover when the needle cover moves towards the retracted position,
blocking means configured to prevent the medical container from moving proximally inside the housing, such that the medical container cannot displace the holder towards the active position, wherein the blocking means include a friction-fit ring which is secured to a locking element configured to lock the needle cover in the second extended position, and the friction-fit ring defines a central opening for frictionally engaging the barrel such that proximal movement of the barrel is prevented.

2. The autoinjector according to claim 1, wherein the friction-fit ring includes radial protrusions protruding from an inner wall of the central opening, said radial protrusions defining an inner diameter lower than an outer diameter of the barrel, and said radial protrusions longitudinally extending in a circumferential direction.

3. The autoinjector according to claim 2, wherein the radial protrusions have a slanted proximal wall for allowing insertion of the barrel in the distal direction through the central opening.

4. The autoinjector according to claim 2, wherein the radial protrusions are made of a resilient material.

5. The autoinjector according to claim 1, wherein the friction-fit ring is configured to define an accommodation cavity between an inner wall of the central opening and the barrel, said accommodation cavity being configured to receive a glue material.

6. The autoinjector according to claim 1, wherein the friction-fit ring includes snap-fitting means allowing a snap-fit connection with a flange of the barrel.

7. The autoinjector according to claim 1, wherein the friction-fit ring is overmolded on the locking element.

8. The autoinjector according to claim 1, wherein the blocking means include a spacer ring defining a central opening for allowing insertion of the plunger rod, the spacer ring being configured to abut on one side against a flange of the medical container, and the spacer ring having radially protruding lugs so as to abut, on the other side, against an abutment surface of the housing.

9. The autoinjector according to claim 8, wherein the lugs have an increasing width in an outward direction.

10. The autoinjector according to claim 8, wherein the abutment surface of the housing is defined by an axial rib of the housing.

11. The autoinjector according to claim 8, wherein the spacer ring has snap-fitting means for removable attachment to the holder.

12. The autoinjector according to claim 11, wherein the snap-fitting means include two, and preferably only two, axial protrusions that are diametrically offset.

13. The autoinjector according to claim 8, wherein the spacer ring has a recessed edge portion for receiving a portion of a locker, said locker being coupled to the holder, and the locker being movable with regard to the housing between a locking position, wherein the locker prevents the holder from moving to the activated position, and an unlocking position, wherein the locker rotates around the longitudinal axis A for allowing the holder to move to the activated position, rotation of the locker from the locking to the unlocking position being caused by the needle cover moving towards the retracted position.
